(19) United States
(12) Patent Application Publication
(10) Pub. No.: US 2004/0243093 A1
(43) Pub. Date: Dec. 2, 2004

(54) System for growth, analysis, storage, validation and distribution of cells and tissues used for biomedical purposes

(76) Inventors: Ron Berenson, Mercer Island, WA (US); Roy H. Hammerstedt, Boalsburg, PA (US); Kenneth N. Sherman, Seatttle, WA (US)

Correspondence Address: WEBB ZIESENHEIM LOGSDON ORKIN & HANSON, P.C., 700 KOPPERS BUILDING, 436 SEVENTH AVENUE, PITTSBURGH, PA, 15219, US

(21) Appl. No.: 10/466,065

(22) PCT Filed: Jan. 10, 2002
(86) PCT No.: PCT/US02/00803

Related U.S. Application Data
(60) Provisional application No. 60/260,728, filed on Jan. 10, 2001.

(51) Int. Cl. G06F 19/00; A61M 37/00; B65D 30/02; A01N 1/02; G01N 33/48; G05B 21/00

(52) U.S. Cl. 604/404; 604/6.15; 435/2; 383/1; 702/19; 700/273

(57) ABSTRACT

A comprehensive system (10) for the growing, analyzing, storing, validating and distributing of cells or tissues for a variety of purposes, in which the comprehensive system (10) generally uses a macrocontainer (12) for each of the steps from beginning to end. The macrocontainer (12) may include a number of elements, an essential one of which is the primary container (14). In every case, the primary container (14) includes a biosensor and a data registry device (15) (usually a microchip) to record and to display the handling history of the primary container (14) throughout the implementation of the system (10). The macrocontainer (14) provides process control, sterility and a matrix within and around which associated inlets, outlets and data lines may be coordinated.

### BACKGROUND OF THE INVENTION

1. Field of the Invention

The invention pertains to a system for growing, storing and distributing cells and tissues useful in medical or other applications.

2. Description of Related Art

Bacterial, yeast, insect and mammalian cells are often isolated, modified and grown for medical and other purposes. Current approaches for the production of cells and tissues for biomedical use involve a “batch process,” whereby large quantities of cells are grown, harvested, analyzed, stored and delivered in a series of discrete steps. In general, transition from one step to the next involves centrifugation, concentration, serial sampling, analysis, etc. Many of these steps are potentially deleterious to cell viability, are difficult to conduct under biosecure conditions and are difficult to reproduce precisely. Such inefficiencies raise production costs and degrade product quality.

Certain U.S. patents identify technologies pertaining to individual steps in the overall growth, analysis, storage, validation and distribution of cells and tissues, but none addresses the development of a single system to accomplish the tasks in an integrated manner. U.S. Pat. No. 6,315,767 to Dumont et al. describes a means to add materials to a sealed bag of platelets, but does not describe exchange of media after that bag is sealed. U.S. Pat. No. 5,261,870, No. 6,124,483 and No. 6,065,294 to Hammerstedt et al. describe a means to add and to remove solutes from a sealed bag in an aseptic manner, but do not address the use of integral sensors to assess when the process should be moved to the next step. Biosensors for use within cell or tissue containers are known, but are not disclosed as components in an overall, preferably automated control system for processes occurring in a biosensor equipped container. Even though on-line monitoring of progress through individual cell or tissue management steps has been considered, see Zeiser et al., “On-line monitoring of the progress of infection in Sf-9 insect cell cultures using relative permittivity measurements,” Biotechnology Bioengineering, vol. 63, pp. 122-126 (1999), the state of the art has apparently not attempted or even appreciated that a single system to manage the start to finish business of growing, analyzing, storing, validating and distributing cells and tissues would provide a useful and medically cost efficient innovation. Documentation of the processes to assure and to verify quality control of each individual step, necessary to assure compliance, is both expensive and labor intensive. If the resultant product must be shipped to sites of use, further documentation is needed to validate authenticity and conditions during transit. A need therefore remains for a start to finish system for growing, analyzing, storing, validating and distributing cells and tissues for various medical purposes, which system embraces maximum automation and minimal container changes.

### SUMMARY OF THE INVENTION

In order to meet this need, the present invention is a comprehensive system for the growing, analyzing, storing, validating and distributing of cells and tissues for a variety of purposes, in which the comprehensive system generally uses the same container(s) for each of the steps from beginning to end. The macrocontainer may include a number of elements, an essential one of which is the cassette, namely, the container which holds the cells or tissue from beginning to end. Another name for the cassette is “primary container.” In every case, the cassette, or primary container, bears a data registry device (usually a microchip) to record and to reproduce the handling history of the primary container throughout the implementation of the system. The macrocontainer encapsulates the cassette in part for the purpose of exchanging fluids into and out of the cassette during processing of the cells or tissue in the cassette, but also to monitor and in many cases to implement process steps as controlled by a microprocessor. Other features of the invention include a biosensor within the cassette, separate from the above-mentioned microprocessor, which biosensor is positioned interior so as to partly extend into the interior of the cassette. In the preferred embodiment of the invention, the biosensor is an integral biosensor, namely, a biosensor integral to the primary container/cassette. Ordinarily, the cassette contains gated and/or ungated pores which cooperate in the treatment of the cells or tissues within the cassette. The macrocontainer provides process control, sterility and a matrix within and around which associated inlets, outlets and data lines may be incorporated and coordinated.

### BRIEF DESCRIPTION OF THE DRAWING(S)

FIG. 1 is a sectional view of the preferred embodiment of the comprehensive system according to the present invention.

### DESCRIPTION OF THE PREFERRED EMBODIMENT(S)

The present invention is a comprehensive system for the growing, analyzing, storing, validating and distributing cells or tissues for a variety of purposes, in which the comprehensive system 10 uses the same container(s) for each of the steps from beginning to end. The macrocontainer may include a number of elements, an essential one of which is the cassette, namely, the container which holds the cells or tissue from beginning to end. Another name for the cassette is “primary container.” In every case the cassette, or primary container, bears a data registry device (usually a microchip) to record and to reproduce the handling history of the primary container throughout the implementation of the system. The macrocontainer encapsulates the cassette in part for the purpose of exchanging fluids into and out of the cassette during processing of the cells or tissue in the cassette, but also to monitor and in many cases to implement process steps as controlled by a microprocessor. Other features of the invention include a biosensor within the cassette, separate from the above-mentioned microprocessor, which biosensor is positioned so as to partly extend into the interior of the cassette. In the preferred embodiment of the invention, the biosensor is an integral biosensor, namely, a biosensor integral to the primary container/cassette. Ordinarily, the cassette contains gated and/or ungated pores which cooperate in the treatment of the cells or tissues within the cassette. The macrocontainer provides process control, sterility, and a matrix within and around which associated inlets, outlets and data lines may be incorporated and coordinated.

For the purpose of the ensuing further description, the following definitions are illustrative.

Integral Biosensor: “Integral Biosensor” designates one of many devices as disclosed herein, which is typically incorporated into a primary container (or cassette), prior to sterilization if appropriate, and separates a biosensor (see below) from the contents of the sealed cassette or primary container yet provides information on quality of the contents of the container without breaking the sealed system.

Gated Pore: In this disclosure, we use the term “gated pore” or “gated pore membrane” in a broad sense, to include any separation barrier containing one or more pores which have been occluded as taught, for example, in U.S. Pat. No. 5,261,870. Such a separation barrier has one or more pores which initially are closed, but which will open in response to a change in the local environment to an extent sufficient to allow passage of ions or molecules, as appropriate. The occluding material might erode, dissolve or change three-dimensional form, depending on the design of the gated pore membrane. Simple erosion of a gated pore upon exposure to an adjacent liquid has widespread utility in the initial compartmentalization, and later fluid treatment, of cells and other biological materials.

Gate: In this disclosure, we use the term “gate” in reference to one or more pores through a separation barrier. The gate is “closed” if the pore is occluded to prevent passage of ions or molecules. The gate is “open” if the occluding material has eroded, dissolved or changed three-dimensional form sufficiently to allow passage of one or more species of selected ions or molecules.

Sensor Compartment: “Sensor Compartment” refers to a component of an integral sensor, with said component formed in part from a gated pore membrane and in part from a plastic construct. The sensor compartment contains and positions a biosensor where it can become accessible to ions, molecules or cells entering from the primary container and also positions that biosensor where it can be interrogated in a consistent manner through a special optical window or, alternatively, through a portion of the primary container or cassette.

Biosensor: “Biosensor” refers to a component of a device, with said component designed to respond to a molecular change, or multiple molecular changes, in the local environment in a known and consistent manner with a response or signal that can be detected or measured using procedures known to those skilled in the art. Typically, the change is in the local environment or the microenvironment, which within the primary container or cassette might be ionic, molecular or cellular in nature.

Recepter: “Receptor” is a term used in reference to any detector molecule, either synthetic or natural in origin, or antibody incorporated into a biosensor. A receptor has reasonable affinity and specificity for one or more “ligands” (see below).

Ligand: “Ligand” is a term used in reference to a specific ion, molecule or cell accumulating or disappearing within a primary container or cassette over time. Accumulation of ligand within the primary container will result in a change in the proportion of unoccupied receptor to ligand receptor complexes, and a change in the characteristics reported by the biosensor.

The present invention deals with a comprehensive system for tracking, monitoring and governing the growth, analysis, storage, validation and distribution of cells or tissues for a wide variety of medical or related applications. The present invention, therefore, incorporates devices and methods which allow evaluation of the cell or tissue contents of a primary container or cassette by means, in part, of an integral biosensor which is separated from the contents of the primary container yet provides information on quality of the contents of the primary container without breaching the system. In addition to the presence and operation of the integral biosensor, a data registry device—usually a microchip—is positioned as a part of or immediately adjacent to the primary container. The purpose of the data registry device is to track and to report times elapsed and all coordinated process criteria (temperatures, locations, etc., through the processing period). In the preferred embodiment, the data registry can be read by an external device, but cannot be altered by devices external to the system.

Ordinarily, the primary container resides partly or completely within a macrocontainer, which is a larger enclosure within and around which additional features of the invention are coordinated. For example, an optional microprocessor may be used to govern process steps to which the primary container is subjected. Optional auxiliary sensor(s) are likewise provided in cooperation with the macrocontainer, which auxiliary sensor(s) are equipped to monitor temperature, pressure, time, global position, light exposure, sound and vibration exposure and/or electromagnetic exposure of the system as a whole. Optional fluid reservoir(s) are also provided so that process steps requiring introduction and removal of fluids may be governed by the microprocessor. The key to the present invention is the combination of the biosensor and the primary container, which primary container is in turn provided with the data registry device, providing a hereinbefore unattainable product in which a biological material may be monitored and documented (verified) without any change of container at any point in processing. For this reason the microprocessor, the auxiliary sensor(s) and the fluid reservoir(s) are nominated as optional although they contribute to an optimal system as a whole, because they facilitate automated processing and documenting of biological materials from collection and growth through analysis, storage, further processing and distribution. The data registry device is designed so as to provide, upon “reading” by the end user, all of the handling and processing information necessary to verify the suitability of the primary container contents for the intended use.

The present invention always incorporates a biosensor, usually an integral biosensor, within the primary container. The integral biosensor is separated from the contents of the primary container but provides information on quality of the contents of the primary container without breaching those contents. The integral sensor device might be a hollow cylinder or a shallow construct. One end or face of the device is a gated pore membrane whose pores normally are occluded, by one of many approaches, forming one end of a sensor compartment, containing a biosensor appropriate for the task, with the other end of the sensor compartment being formed by an optical window recessed in from the end opposite to the gated pore membrane or formed by a wall of the primary container. Typically, the integral sensor device is fabricated separately from the primary container and incorporated into the primary container during final fabrication, before sterilization. Certain embodiments of the device are therefore capable of aseptic operation.

The status of the contents of a primary container for blood cells, other cells, foods or industrial products can be determined by inspection, visually or via a fiberoptic probe through the optical window of a plastic construct incorporated into said primary container at fabrication. When the biosensor is retained within the plastic construct by a gated pore membrane, the pores of which open in response to an environmental change in the primary container, the contents of the primary container can contact and cause a change in the biosensor. Alternatively, a cell suspension within a primary container may undergo a change which itself signals gated pore membranes to open, and in turn fluid enters the biosensor upon opening of the gated pore membrane.

Changes in the primary container which can be detected include, but are not limited to, either a decrease or an increase in pH away from a threshold value, or accumulation of one or more members of a preselected class of molecules, including toxins produced by bacteria, above a threshold value. A great range in utility is possible because, depending on the device and method, both the material(s) occluding the gated pore membrane and material(s) forming the biosensor can be varied independently or in combination. Hence, a predetermined change in contents of the primary container can be evidenced by opening of the gated pore membrane and/or a change in the signal from the biosensor. A typical biosensor function would be to detect unwanted bacteria, at a level of, for example, one bacterium per 10,000 platelets as a single illustration.

Conventional membranes with pores of known size (i.e., 0.1 or 3.0 micrometer nominal diameter) can be fabricated into special gated pore membranes, and then fabricated into segments of the components of the present invention (see U.S. Pat. No. 5,261,870). The gated pore membrane can provide a closed container which opens only when predefined conditions are met, and the material occluding or otherwise closing the pores is altered so that the gates previously blocking passage of molecules through the pores are opened. The pore plugging material may thus simply erode at a predefined rate upon exposure to water, or may require a particular pH or other environmental condition to initiate erosion of the pore plugs. An almost infinite combination of membranes, pore diameters and occluding materials is envisioned in U.S. Pat. No. 5,261,870. Importantly, the conditions on one side of the gated pore membrane and not mechanical, electrical or other interventions determine when the pores open and allow the passage of molecules through the membrane. Any membrane stock may be used for the gated pores, namely, fibril membranes with “haystack” structure, membranes with “tunnel structure,” or stock membranes with built-in ability to respond to pH. In the present invention, both the gated pores and the integral biosensor address the contents of the primary container and not the overall reaction conditions. There is no limit to biosensors appropriate for use within the present system, as long as they undergo a perceptible change in character upon exposure to a predetermined environmental change to be monitored. Detector substances or molecules may be combined with substrates, such as plastic beads and other substrates or carriers, known in the art.

Biosensors can be designed to respond to many molecular changes in the environment. The color change of a pH indicator in response to proton concentration or certain dipsticks to glucose concentration are two common and simple examples. Biosensors sensitive to one or another molecular stimulus can be incorporated into beads or micro-beads, and frequently can be designed to change color or to emit light of a given wavelength when exposed to light of an appropriate wavelength such as fluorescence. These or other changes can be monitored with a variety of detectors, ranging from the human eye to fiberoptic electronic devices with a digital readout. Fiberoptic probes may be integral, or separable and positioned near the optical window; in any case they extend to the optical window, but extend to the optical window and not through it. Walls forming the integral biosensor, except for the optical window, may be made of opaque or translucent material to enhance visibility of biosensor reaction through the optical window.

It should be apparent from the foregoing that whereas the integral biosensor interrogates changes only within the cells or tissue and other contents of the primary container, the auxiliary sensor(s) and the microprocessor are concerned with the macrocontainer and the overall process conditions. The data registry device records all data.

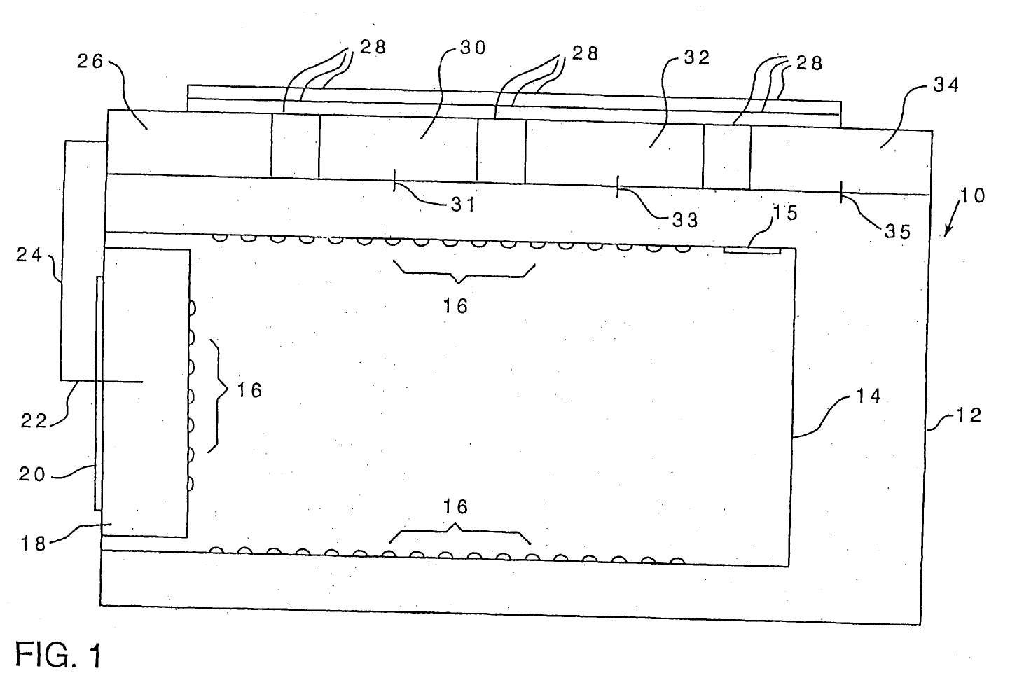

Referring now to FIG. 1, the comprehensive system 10 includes a macrocontainer 12 which surrounds a primary container 14 containing gated pores 16, with the primary container 14 being constructed so as to incorporate an integral biosensor 18. The integral biosensor 18, in this preferred embodiment of the invention, is positioned so that a single wall of the primary container 14 and the macrocontainer 12 integrally provide the optical window 20 and allow it to be viewed by the associated fiberoptic probe 22. The fiberoptic probe 22 may be permanently mounted or may be removable with respect to the optical window 20. The side walls of the integral biosensor 18 may be opaque, translucent or fluorescent. All processing of and within the macrocontainer 12 is governed by the microprocessor 26, which via data lines 28 and the fiberoptic probe data line 24 has access to data and can monitor and govern process conditions and steps. Optional auxiliary sensor(s) 30, a first fluid reservoir 32 and a second fluid reservoir 34 operate in conjunction with the microprocessor 26 by way of the auxiliary sensor probe(s) 31, the first fluid reservoir inlet/outlet 33, the second fluid reservoir inlet/outlet 35, and any other fluid reservoirs or inlet/outlets or other tubes commensurate in design to those illustrated. The inlet/outlet(s) to/from a fluid reservoir could, but need not, incorporate gated pore barriers or membranes. Not shown in FIG. 1 are the data lines which connect the data registry device 15 to any or all of the integral biosensor 18, the fiberoptic probe 22, the microprocessor 26, the auxiliary sensor(s) 30 or the reservoir components. The data registry device 15 is configured to record any data generated by any other component of the system 10. Also not shown in FIG. 1 is the sealable port in the primary container 14 for inoculations and/or removal of contents.

In operation, the system 10 may be implemented as follows. The primary container 14 could be considered, among many other examples, as the “bag” for a quantity of T cells intended to be collected and transformed (activated) prior to administration to a patient. This example is illustrative only. After collection, depending on the length of time and need for transportation, cryopreservation agents may be introduced and removed from the primary container 14 via the gated pores 16, after introduction and/or dilution/removal of cryoprotectants from one or more fluid reservoirs 32, 34. Transition of cryoprotectant through the gated pores 16 can begin, for example, simply by contacting the gated pores 16 with the cryoprotectant solution to initiate erosion. In instances where freezing is not necessary, storage solutions designed to preserve cell viability may be substituted for cryopreservation agents, and may be added and removed via the reservoirs 32, 34 and as governed by the microprocessor 26. Before or after storage and transportation, the T cells may be activated by introducing transforming factors, again through one of the reservoirs 32, 34 as governed by the microprocessor 26. Throughout the entire treatment cycle, the microprocessor 26 and the auxiliary sensor(s) 30 provide all other control functions—monitoring of temperature, verification of extent and length of freezing, if any, fiberoptic assessment (absence of unwanted pH change, bacteria, etc.), determination of location (global positioning) throughout treatment, distribution and thawing, and any other parameter programmed into the auxiliary sensors 30 and the microprocessor 26. If additives for infusion are required prior to patient administration, those additives may originate in yet a further reservoir as governed by the microprocessor 26 as to timing and amounts. The data registry device 15 records all process parameters for the length of treatment from start to finish.

With the tracking of T cells from initial “bag” inoculation through analysis, storage, distribution and administration as exemplary, the adaptability of the present system to other biological materials may easily be envisioned. Gene therapy materials, replacement tissues of all kinds, other blood cells, and other cells or tissues may be inoculated into the primary container and grown and distributed to the end user by any protocol imaginable, with the microprocessor governing the functions and with the data registry device providing a record for verification purposes. Process steps may, therefore, include without limitation, sustaining cells immediately after collection, modifying cells as needed, expanding cell number or growing certain tissues or tissue forms, verifying the attaining of certain cell properties, storing, delivering, infusion preparing, and validating the cells, tissues or other materials of interest. All of these goals may be achieved without ever removing the cells or tissues from the primary container “home”, at least until the final moment of use of the cells or tissues. The system thus provides enormous efficiency because cells or tissues remain in the primary container from growth/collection to final distribution, regardless of distance or storage conditions.

It should be apparent from the foregoing that all except the primary container and the integral biosensor may be configured as reusable constructs, which may be resterilized for use throughout an entire biological cycle from cell growth/maintenance to ultimate distribution. Alternatively, the system can be configured to be reusable except for the primary container and the macrocontainer per se, which can be manufactured as plastic disposable components. Ordinarily, but not necessarily, the data registry device will be a chip physically integral with the primary container and thus ordinarily a disposable component.

Although the invention has been disclosed in terms of particular materials and methods above, the invention is to be limited only insofar as is set forth in the accompanying claims.

The invention claimed is:

1. A biological container and distribution system, comprising a primary container incorporating one or more gated pores, a biosensor and a data registry device.

2. The biological container and distribution system of claim 1, wherein said biosensor is an integral biosensor and said primary container is disposed within a macrocontainer having a processor associated therewith.

3. The biological container and distribution system of claim 2, wherein said integral biosensor is positioned adjacent an optical window in said primary container, and further wherein said processor is a microprocessor.

4. The biological container and distribution system of claim 3, wherein said integral biosensor and said macrocontainer share a wall within which said optical window is disposed.

5. The biological container and distribution system of claim 4, wherein said optical window has a fiberoptic probe associated therewith.

6. The biological container and distribution system of claim 5, wherein said fiberoptic probe is integrally formed with respect to said optical window.

7. The biological container and distribution system of claim 5, wherein said fiberoptic probe is removably formed with respect to said optical window.

8. The biological container and distribution system of claim 7, wherein said macrocontainer includes inlet/outlets thereto.

9. The biological container and distribution system of claim 8, wherein said macrocontainer cooperates with at least one auxiliary sensor.

10. The biological container and distribution system of claim 9, wherein said macrocontainer is juxtaposed among more than one auxiliary sensor and said microprocessor and at least one reservoir.

11. A process for preparing, transporting and distributing cells or tissues, comprising the steps of providing a primary container with gated pores, a data registry device and a biosensor; inoculating said primary container with the cells or tissues to be distributed, storing and transporting said primary container under predetermined conditions, verifying the cell or tissue conditions by means of said data registry device, and distributing the contents of said primary container after verification is complete.

12. The process of claim 11, wherein said process is governed by a processor.

13. The process of claim 12, wherein said processor is a microprocessor.

14. The process of claim 13, wherein said auxiliary sensors monitor at least one of temperature data, pressure data, time, global positioning data, light exposure data, sound and vibration exposure data, and/or electromagnetic exposure data, and further wherein said data is communicated to said data registry device.

15. The process of claim 14, wherein said data registry device also receives data from said biosensor, which further is an integral biosensor.

16. The process of claim 15, wherein said microprocessor is operationally interconnected with said integral biosensor and said data registry device by means of a fiberoptic probe.
